(19) United States
(12) Patent Application Publication
(10) Pub. No.: US 2015/0353399 A1
(43) Pub. Date: Dec. 10, 2015

(54) COMPOSITIONS, DEVICES, AND METHODS INVOLVING DEGRADATION OF CYANURIC ACID

(71) Applicant: REGENTS OF THE UNIVERSITY OF MINNESOTA, Minneapolis, MN (US)

(72) Inventors: Lawrence Philip Wackett, St. Paul, MN (US); Alptekin Aksan, Minneapolis, MN (US); Baris Ragip Mutlu, Minneapolis, MN (US); Sujin Yeom, Falcon Heights, MN (US); Adi Ish Am Radian, Falcon Heights, MN (US); Kelly Aukema, Mendota Heights, MN (US)

(21) Appl. No.: 14/735,574

(22) Filed: Jun. 10, 2015

Related U.S. Application Data
(60) Provisional application No. 62/010,282, filed on Jun. 10, 2014.

(51) Int. Cl. C02F 3/34; C12N 1/20

(52) U.S. Cl. CPC C02F 3/342; C12N 1/20; C02F 2101/38

(57) ABSTRACT

This disclosure describes compositions, devices, and methods relating to removal of cyanuric acid from a sample. Generally, the compositions include a cell that expresses at least one enzyme that degrades cyanuric acid and a matrix material that covers at least a portion of the cell. Generally, the methods include contacting the sample with the composition, or a device that includes the composition, and allowing the enzyme of the composition to degrade cyanuric acid in the sample.

### CROSS-REFERENCE TO RELATED APPLICATION

This application claims priority to U.S. Provisional Patent Application Ser. No. 62/010,282, filed Jun. 10, 2014, which is incorporated herein by reference.

### GOVERNMENT FUNDING

This invention was made with government support under 1237754 awarded by the National Science Foundation. The government has certain rights in the invention.

### SEQUENCE LISTING

This application contains a Sequence Listing electronically submitted to the United States Patent and Trademark Office via EFS-Web as an ASCII text file entitled “2015-06-10-SeqList454ST25.txt” having a size of 5 kilobytes and created on Jun. 10, 2015. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY

This disclosure describes, in one aspect, a composition that generally includes a cell that expresses at least one enzyme that degrades cyanuric acid and a matrix covering at least a portion of the cell. In some cases, the cell can be genetically-modified to exhibit an increase in cyanuric acid degradation activity compared to a wild-type control.

In some embodiments, the enzyme can be a cyanuric amidohydrolase.

In some embodiments, the enzyme can be a cyanuric acid hydrolase.

In some embodiments, the matrix can include a chlorine-reactiver material such as, for example, an amine-containing material or a thiol-containing material.

In another aspect, this disclosure describes devices for removing cyanuric acid from a sample. Generally, the devices include an embodiment of the composition summarized above.

In another aspect, this disclosure describes methods for removing cyanuric acid from a sample. Generally, the methods include contacting the sample with any embodiment of the composition summarized above and allowing the enzyme of the composition to degrade cyanuric acid in the sample.

In some embodiments, the sample can include chlorinated water. In some of these embodiments, the sample can include hypochlorite ion.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Reaction of cyanuric acid and hypochlorite ion that occurs in an excess of cyanuric acid.

FIG. 2. AtzD activity remaining after 15 minute incubation times of the enzyme with the indicated concentrations of trichlorocyanuric acid as a source of chlorine.

FIG. 3. Cyanuric acid degradation (ammonia production) over time using various silica-encapsulated materials.

FIG. 4. Cyanuric acid degradation (ammonia production) over time using uncoated free cells.

FIG. 5. Cyanuric degradation in the absence (left) or presence (right) of 2.0 ppm hypochlorite ion by APTES-encapsulated cells.

FIG. 6. Cyanuric degradation in the absence (left) or presence (middle) of 50 ppm hypochlorite ion by APTES-encapsulated materials.

FIG. 7. CAH structure with barbituric acid bound (center).

FIG. 8. Cyanuric acid hydrolase. (A) Entire active site cavity of cyanuric acid hydrolase with barbituric acid in the enzyme's reactive site. (B) Amino acid sequence alignment of selected regions of the enzyme showing conservation of amino acid residues.

FIG. 9. Minimal active site model showing serine poised for nucleophilic attack on barbituric acid.

FIG. 10. Stoichiometry of CAH-protective reactions in exemplary CAH-protective gels.

FIG. 11. Chemical structure of 3-aminopropyltriethoxysilane (APTES).

FIG. 12. Data comparing protection of cyanuric acid hydrolase activity against chlorine inactivation by various silica-covered composite materials.

FIG. 13. Standard curve showing absorbance as a function of cyanuric acid concentration.

FIG. 14. Reaction cycle of cyanuric acid hydrolase.

FIG. 15. (A) Hydrolysis of 3-aminopropyltriethoxysilane (APTES). (B) schematic diagram illustrating disruption of outer membrane by APTES.

FIG. 16. Cyanuric acid hydrolase activity is protected against hypochlorite by APTES gel. Bleach (hypochlorite) was added one hour prior to adding 140 ppm cyanuric acid.

FIGS. 17. (A) and (B) APTES-encapsulated cells allow “leaking” of small molecules while retaining cyanuric acid-degrading enzymes. (C) Scanning electron micrograph images of an E. coli cell expressing cyanuric acid hydrolase after TEOS-encapsulation. (D) An E. coli cell after removal from APTES gel encapsulation.

FIG. 18. Cyanuric acid concentration measured over time following three different treatments: () E. coli (CAH) encapsulated in TEOS gel; (□) E. coli (CAH) cells suspended without silica; (▴) Cell-free cyanuric acid hydrolase enzyme.

FIG. 19. Rates of cyanuric acid degradation by APTES-encapsulated E. coli (CAH) and APTES-coated E. coli (CAH) (APTES Treatments, right) compared to TEOS-encapsulated cells, suspended cells, and free enzyme (Controls, left).

FIG. 20. Cyanuric acid concentration following four different treatments: APTES-coated E. coli (CAH) cells, no hypochlorite; E. coli (CAH) cells suspended without silica, 10 ppm hypochlorite; APTES-encapsulated E. coli (CAH) cells, 10 ppm hypochlorite; APTES-partially-coated E. coli (CAH) cells, 10 ppm hypochlorite.

FIG. 21. Data showing chlorine removal by crumb rubber.

FIG. 22. Data showing chlorine removal by cherry wood sawdust.

### DETAILED DESCRIPTION OF ILLUSTRATIVE EMBODIMENTS

This disclosure describes compositions, devices, and methods that involve an enzyme-based treatment for removing cyanuric acid from a fluid system. Cyanuric acid is a chemical that is extensively used in conjunction with hypochlorite and/or chlorinated isocyanuric acids for disinfection. The general use of the enzyme cyanuric acid hydrolase for removing cyanuric acid from a fluid system is described in U.S. Pat. No. 8,367,389. This disclosure describes, however, using the enzyme in a whole cell biological composition.

In many conventional scenarios where one may contemplate using an enzymatic process to control cyanuric acid levels, hypochlorite anion (commonly referred to as bleach or chlorine) is present along with the cyanuric acid. Enzymes that degrade cyanuric acid (e.g., cyanuric acid hydrolase) can, however, be inhibited by even small amounts of bleach and/or chlorinated isocyanuric acid compounds. Thus, the co-presence of cyanuric acid with compounds that inhibit cyanuric acid-degrading enzymes presents challenges for using enzyme-based approaches for controlling cyanuric acid levels.

In contrast, the compositions, devices, and methods described herein can provide sufficient protection to the enzyme to allow the enzyme to remove cyanuric acid in the presence of hypochlorite anion or chlorinated isocyanuric acids. For example, this makes possible the use of a pool filtration material that includes a whole cell composition that can be used continuously while a water system (e.g., a pool) is disinfected by a chlorine disinfectant. The compositions, devices, and methods described herein extend the utility of enzyme-based treatment systems for most commercial applications.

Cyanuric acid, bleach, and/or chlorinated isocyanuric acids (e.g., dichloroisocyanuric acid and trichloroisocyanuric acid) are used, for example, in swimming pools, spas, or wastewater treatment as a disinfectant; in the textile industry as bleaching compounds; and in veterinary treatments in husbandry and fisheries. A common use of these compounds is for swimming pool chlorination. Many of these compounds dissolve slowly in water, thereby allowing a continuous metered dosing of chlorine.

While described in the context of an exemplary embodiment in which the control of cyanuric acid levels is desired in a particular water system (e.g., a swimming pool), the compositions, devices, and methods described herein can provide control of cyanuric acid levels in any suitable context. Exemplary other contexts include, for example, other water systems (e.g., spas, fountains, cooling waters, wastewater treatment, etc.), food treatment, textile bleaching, and husbandry and fishery health.

A common issue in long-term disinfection with bleach is that the hypochlorite anion can react and be irreversibly converted to salt (chloride), chlorate anion, and/or oxygen gas. While these reaction products are not dangerous, the disinfection activity of the hypochlorite is diminished as the hypochlorite is irreversibly converted to these other products. Particularly in the context of outdoor swimming pools, sunlight can hasten these degradation reactions, potentially exposing people using the swimming pool to disease-causing microorganisms (e.g., viruses, bacteria, and the intestinal parasite Giardia) that would otherwise be killed if the hypochlorite were still present at effective levels. One conventional solution to the problem of hypochlorite stability is to provide hypochlorite and cyanuric acid in the pool together. Cyanuric acid serves to stabilize the hypochlorite, thereby reducing the rate and extent to which the hypochlorite is degraded, and thereby extending the effective life of hypochlorite treatment of, for example, a swimming pool.

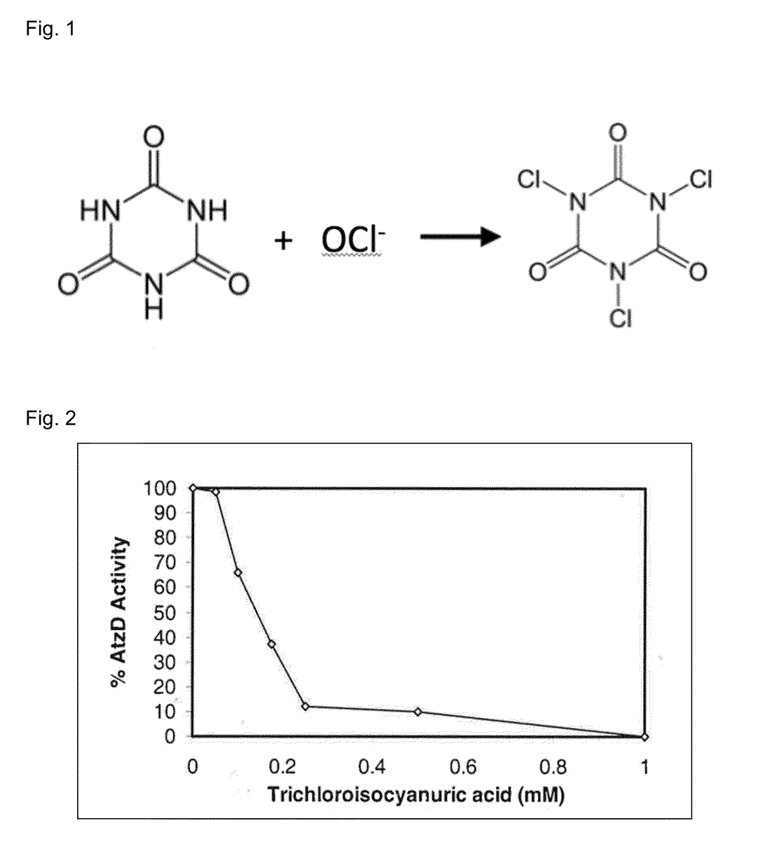

This conventional strategy has its limitations, however. As chlorinated isocyanuric acids (e.g., dichloroisocyanuric acid and trichloroisocyanuric acid) are added to, for example, a swimming pool, cyanuric acid builds up in the pool. When the cyanuric acid concentration is high (e.g., >120 ppm), then the disinfection activity decreases. The chemical equilibrium favors the chlorine atom of the hypochlorite ion reacting with a nitrogen atom of cyanuric acid, as shown in FIG. 1. When the cyanuric acid levels are high enough, the pool needs to be drained of water and refilled. Cyanuric acid can be difficult to remove from swimming pools without draining the water because cyanuric acid is a very stable compound. Thus, chemicals that destroy cyanuric acid tend to have properties that make them unfit for exposure to humans and, therefore, unfit for an application that involves simply adding the cyanuric acid-destroying chemical to a swimming pool.

Enzyme catalysis offers a human-tolerable way to destroy the cyanuric acid ring by catalyzing the addition of water to the ring structure of cyanuric acid so that the nitrogen atoms of the cyanuric acid ring no longer react with the disinfecting hypochlorite ions in the pool water. Adding enzyme directly to the pool can be expensive. A more cost-effective method involves immobilizing the enzyme onto a solid support and have the pool water pass over that support. Since pool water is filtered routinely to remove particles, an enzyme-functionalized surface of a pool filtration device is one exemplary way to deploy such an enzyme.

This disclosure describes, generally, compositions and methods that involve enzymatic catalysis of cyanuric acid. The compositions generally include a cell that expresses an enzyme that can degrade cyanuric acid. The cell is at least partially coated—and in some embodiments may be fully encapsulated—with a silica-based matrix material that includes a chemical group that reacts with bleach (e.g., hypochlorite, chlorine, trichloroisocyanuric acid, etc.). The chemical group of the matrix material can react with the bleach, thereby reducing the extent to which the bleach inactivates the cyanuric acid-degrading enzyme. As described in more detail below, the chemical group that can react with bleach can include an amine, a thiol, a thioether, a phenol, or the like.

The enzyme may be any enzyme that suitably breaks down cyanuric acid. Enzymes that break down cyanuric acid include, for example, cyanuric amidohydrolase (TrzD, Karns, J. S., 1999, Appl. Environ. Microbiol. 65:3512-3517) and cyanuric hydrolases such as, for example, AtzD originally identified from Pseudomonas sp. ADP (Fruchey et al., 2003, Appl. Environ. Microbiol. 69:3653-3657), the cyanuric acid hydrolase (CAH) originally identified from Bradyrhizobium japonicum USDA 110, the CAH originally identified from Rhizobium leguminosarum bv. viciae 3841, the CAH originally identified from Methylobacterium sp. 4-46, and the CAH originally identified from Azorhizobium caluindolans ORS 571 locus AZC_{—}3892. (Seffernick et al., 2012, J. Bacteriol. 194:4579-4588), and an approximately 40 kDa thermostable cyanuric acid hydrolase from Moorella thermoacetica (gi83590946, locus tag Moth 2120 from M. thermoacetica ATCC 39073; Li et al., 2009, Appl. Environ. Microbiol. 75:6986-6991 and U.S. Pat. No. 8,367,389).

Additional suitable enzymes include cyanuric acid amidohydrolase from each of the following sources: Acidithiobacillus thiooxidans, Agrobacterium rhizogenes, Agrobacterium vitis, Alcanivorax dieselolei, Alicychphilus sp. CRZ1, Arthrobacter sp. AD25, Azorhizobium caulinodans, Azorhizobium doebereinerae, Belnapia sp. F-4-1, Belnapia moabensis, Bradyrhizobium sp. Ai1a-2, Bradyrhizobium sp. ARR65, Bradyrhizobium sp. BTAi1, Bradyrhizobium diazoefficiens, Bradyrhizobium sp. DOA9, Bradyrhizobium sp. Ec3.3, Bradyrhizobium elkanii, Bradyrhizobium japonicum, Bradyrhizobium sp. LTSP885, Bradyrhizobium sp. LTSPM299, Bradyrhizobium oligotrophicum, Bradyrhizobium sp. ORS 278, Bradyrhizobium sp. ORS 285, Bradyrhizobium sp. ORS 375, Bradyrhizobium sp. S23321, Bradyrhizobium sp. STM 3809, Bradyrhizobium sp. STM 3843, Bradyrhizobium sp. URHA0013, Bradyrhizobium sp. WSM471, Bradyrhizobium sp. WSM1253, Bradyrhizobium sp. WSM1417, Bradyrhizobium sp. WSM1743, Bradyrhizobium sp. WSM2254, Bradyrhizobium sp. WSM3983, Bradyrhizobium sp. YR681, Bradyrhizobium yuanmingense, Clostridiales bacterium VE202-15, Clostridium ultunense, Clostridium asparagiforme, Comamonas sp. A2, Derxia gummosa, Gluconacetobacter sp. SXCC-1, Mesorhizobium sp. WSM4349, Methylobacterium sp. 285MFTsu5.1, Methylobacterium aquaticum, Methylobacterium sp. B1, Methylobacterium sp. B34, Methylobacterium oryzae, Methylobacterium sp. UNCCL110, Oceanicola sp. 22II-S11g, Oceanicola granulosus, Paenibacillus borealis, Paenibacillus daejeonensis, Paenibacillus sp. FSL P4-0081, Paenibacillus sp. FSL R7-277, Paenibacillus sp. FSL R5-0912, Paenibacillus sp. FSL R7-0273, Paenibacillus sp. G4, Paenibacillus sp. JDR-2, Pseudacidovorax intermedius, Pseudomonas aeruginosa, Pseudomonas sp. AK_cAN1, Pseudomonas sp. EGD-AK9, Pseudomonas pelagia, Pseudomonas pseudoalcaligenes, Proteiniclasticum ruminis, Rhizobium sp. 2MFCol3.1, Rhizobium sp. 42MFCr.1, Rhizobium sp. CCGE 510, Rhizobium sp. CF394, Rhizobium galegae, Rhizobium gallicum, Rhizobium sp. LC145, Rhizobium leguminosarum, Rhizobium sp. OV201, Rhizobium tropici, Rhizobium sp. YR519, Roseomonas aerilata, Rubritepida flocculans, Salinisphaera shahanensis, Skermanella aerolata, Skermanella stibiiresistens (e.g., S. stibiiresistens SB22), Variovorax paradoxus, Xanthobacter sp. 91, Xanthobacter sp. 126,

Additional suitable enzymes include the following enzymes from the indicated natural sources: a cyanuric acid hydrolase from Rhodococcus sp. Mel; a barbiturase (atzD) from uncultured marine thaumarchaeote KM3_{—}67_E04, Gloeocapsa sp. PCC 7428, Methylobacterium sp. ME121; a ring-opening amidohydrolase from Bradyrhizobium sp. CCGE-LA001, Pandoraea sp. SD6-2, Nakamurella multipartita DSM 44233, Acidithiobacillus ferrivorans, Celeribacter sp. P73, Acidithiobacillus caldus, or Sulfobacillus acidophilus TPY; or an amidohydrolase from Acidithiobacillus ferrivorans, Algiphilus aromaticivorans, Acidithiobacillus thiooxidans, or Gordonia sp. KTR9.

While described herein in the context of an exemplary embodiment in which the enzyme that breaks down cyanuric acid is a cyanuric acid hydrolase (e.g., AtzD or TrzD), the compositions, devices, and methods described herein can involve the use of any suitable enzyme that breaks down cyanuric acid, whether expressly listed above or not. More than one hundred cyanuric acid hydrolases have been identified, many of which have been characterized. Moreover, the polynucleotides that encode the enzymes have been sequenced.

As noted above, the cost of adding an enzyme to, for example, a swimming pool to degrade cyanuric acid can be cost prohibitive. In addition, cyanuric acid hydrolase can be highly sensitive to trichloroisocyanuric acid and/or hypochlorite anion. Since the pool environment is one where those materials are frequently used for disinfection in order to maintain a safe environment for human use, using cyanuric acid hydrolase to remove cyanuric acid from pools—or anywhere else that chlorine (bleach or hypochlorite or chlorinated isocyanuric acids) are present—remains a challenge.

This disclosure describes compositions in which an enzyme for breaking down cyanuric acid—e.g., a cyanuric acid hydrolase or other suitable enzyme such as those listed above—can be protected from inactivation by trichloroisocyanuric acid and/or hypochlorite anion when the enzyme is expressed from a cell at least partially covered by a matrix material (e.g., a silica-containing gel or sol). As used herein, the term “partially covered” and variations thereof refer to a cell with at least a portion of its outer membrane in contact with the matrix material. The matrix material may continuously or discontinuously cover at least a portion of the cell. In some embodiments, the cell may be encapsulated within a matrix material. As used herein, “encapsulating,” “encapsulated,” and variations thereof refer to a cell that is substantially or completely enclosed within a matrix material. Encapsulating microbial whole cells within a silica microsphere used for the removal of the herbicide atrazine has been previously described (e.g., Reatugui et al. 2012, Appl. Micro. Biotech. 96:231-240 and International Publication No. WO 2012/116031 A2). However, there is no previous report of a synthetic silica matrix material that can protect cells and their cytoplasmic enzymes against the antimicrobial effects of chlorine.

This disclosure therefore describes compositions, devices, and methods that involve removing cyanuric acid from a water system—or other suitable application—using a cell that expresses an enzyme that breaks down cyanuric acid and is at least partially covered with a silica matrix material. The silica matrix material includes a chemical group that can react with bleach or other disinfectant source of chlorine (e.g., hypochlorite, trichlorocyanuric acid, etc.). In some embodiments, a composition that includes the cell at least partially covered with a silica matrix material can be a component of a device that removes cyanuric acid from a source of cyanuric acid (e.g., a water system or other suitable application). In some cases, the device can include one or more additional components that chlorinate and/or filter water. Such a device can simultaneously chlorinate, filter, and remove cyanuric acid from the water source.

A composition that includes the cell at least partially covered with a silica matrix material, as described herein, can allow one to simultaneously chlorinate a water source while maintaining cyanuric acid at level that maintains the chlorine but does not inhibit disinfection (e.g., around 20-40 ppm cyanuric acid). The composition can perform continuous, slow degradation of cyanuric acid rather than conventional pool treatment processes that can require hours.

As noted above, enzymes that degrade cyanuric acid can be sensitive to chlorine. Incubating such an enzyme in a medium that contains chlorine for as little as 15 minutes can be enough to reduce a significant portion—and in some cases, almost all—of the enzymatic activity. FIG. 2 reflects AtzD activity remaining after a 15-minute incubation of enzyme with the indicated concentrations of trichlorocyanuric acid as a source of chlorine. When trichloroisocyanuric acid was present for hours (data not shown), enzyme activity became immeasurably low. Thus, commercial use of an enzyme that breaks down cyanuric acid in the presence of a chlorinating agent seemed unlikely to successfully control cyanuric acid levels.

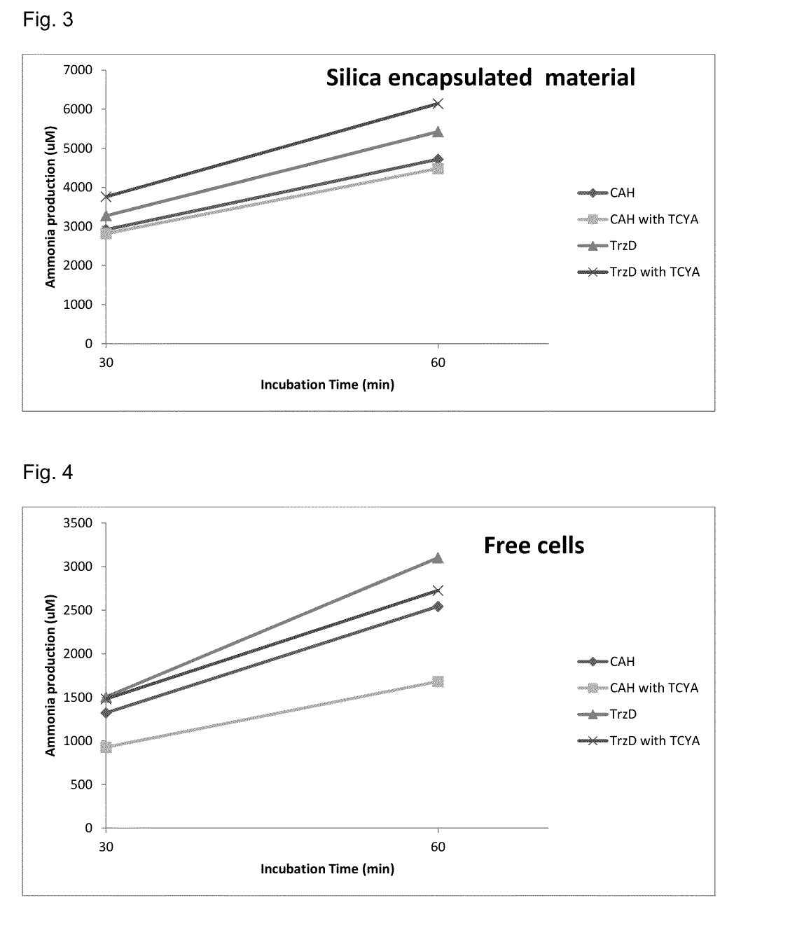

In contrast, E. coli cells that express a cyanuric acid-degrading enzyme and that are at least partially covered with a silica matrix material as described herein—i.e., that possesses a chemical group that can react with chlorine—provided a resilient source of the enzyme in the presence of chlorine. FIG. 3 shows ammonia production as a proxy for cyanuric acid degradation over time using various silica-encapsulated materials. A cyanuric acid and chlorine aqueous solution was treated with the indicated silica-encapsulated biocatalysts (cyanuric acid hydrolases): TrzD and CAH (CAH is described in, for example, U.S. Pat. No. 8,367,389). The cyanuric acid/chlorine mixture contained 3 ppm trichloroisocyanuric acid (TCYA) as a source of chlorine, a level of chlorine that corresponds to chlorine levels used in swimming pools. For each encapsulated biocatalyst, the rate of cyanuric acid degradation is undiminished in the presence of TCYA, showing protection of the enzyme against inactivation.

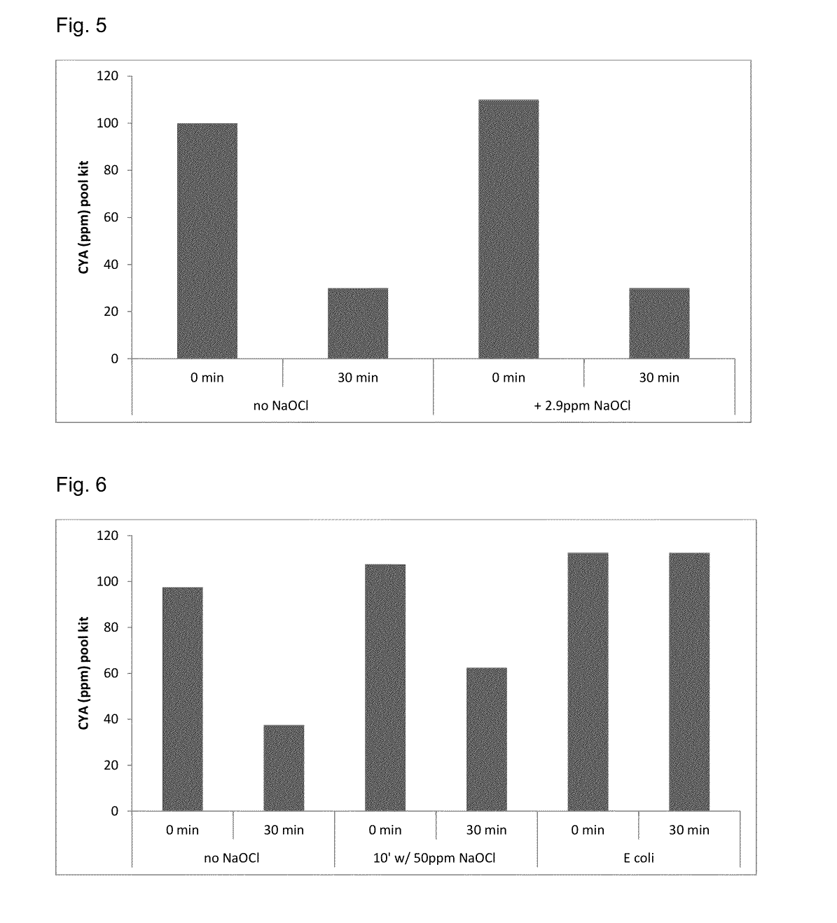

Similar experiments were performed using bleach. Typically, TCYA generates bleach in water, but in the experiments described below, sodium hypochlorite (which is often used to disinfect pools when TCYA is not used) was added directly to the water system. The presence (FIG. 5, right) or absence (FIG. 5, left) of 2.9 ppm sodium hypochlorite provided no discernible difference in cyanuric acid degradation. In this experiment, the bleach had been incubated with the encapsulated biocatalyst for one hour prior to the degradation test, so the biocatalytic material was resilient to exposure to bleach even in the absence of cyanuric acid.

In a subsequent experiment, bleach was added to 50 ppm, a concentration much higher than would typically be used in a pool, to evaluate possible damage to the enzyme at very high concentrations. FIG. 6, middle, shows that 50 ppm bleach diminished biodegradation of cyanuric acid by about 50%.

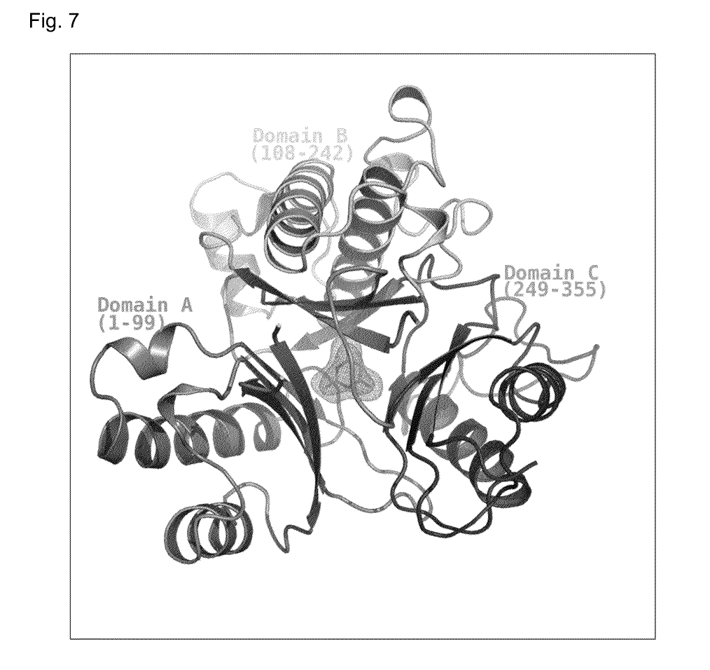

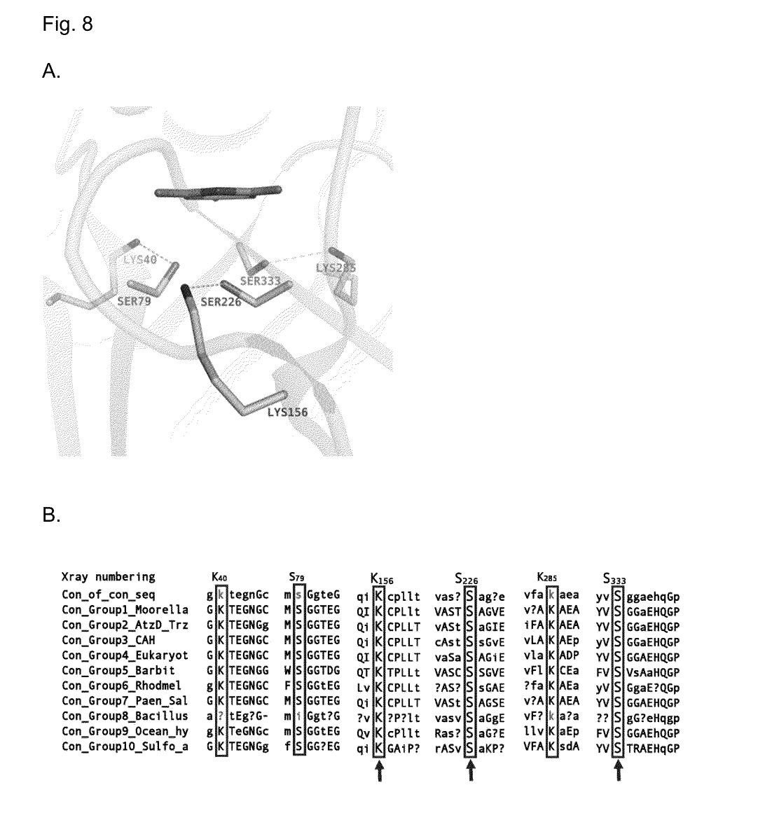

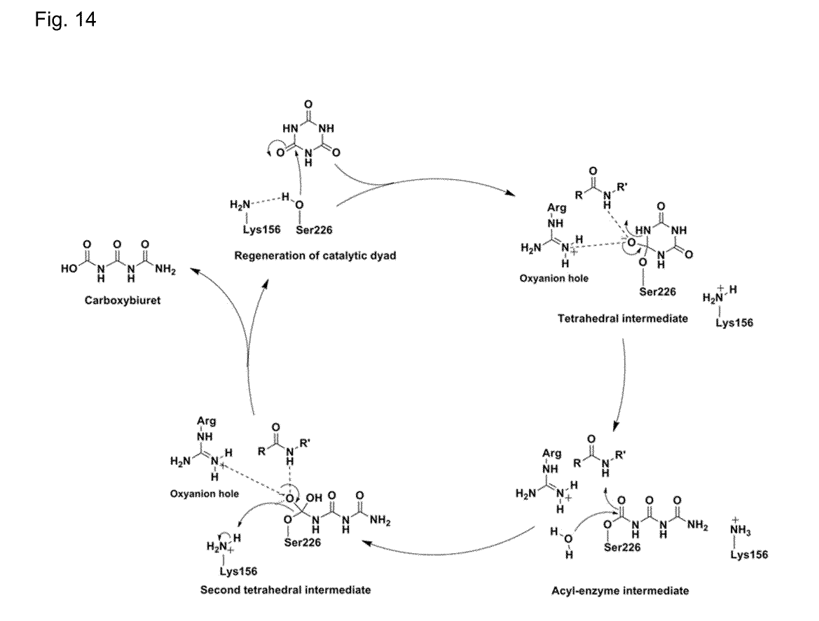

Without wishing to be bound by any particular theory of operation, cyanuric acid hydrolase (CAH, an exemplary cyanuric acid-degrading enzyme) maintains a three-dimensional structure that forms and maintains an active site that has amino acid residues involved in the catalytic activity for degrading cyanuric acid. Cyanuric acid is a stable molecule that does not degrade by itself. Amino acid residues of CAH that are involved in degrading cyanuric acid were identified by determining the three-dimensional location of all 5,373 atoms of the CAH enzyme in the native properly-folded enzyme. The enzyme active site was determined by solving the structure of CAH with barbituric acid, a substrate analog of cyanuric acid, bound in the active site. The CAH protein fold contains three structurally homologous domains forming a β-barrel-like structure with external α-helices that result in a three-fold symmetry, a dominant feature of the structure and active site that mirrors the three-fold symmetrical shape of the substrate cyanuric acid. (FIG. 7). The active site structure of CAH shows three pairs of active site Ser-Lys dyads. (FIG. 8). In order to determine the role of each Ser-Lys dyad in catalysis, a mutational study (Example 2) using a highly sensitive, enzyme-coupled assay (Assay 1, EXAMPLES) was conducted. The 10⁹-fold loss of activity by the S226A mutant resulted in activity that was less than one-tenth of the activity exhibited by the S79A and S333A mutants. In addition, bioinformatics analysis revealed the Ser226/Lys156 dyad as the only absolutely conserved dyad in the CAH enzyme family. There is also a ring of glutamic acid residues and arginine residues involved at the active site. The latter serve as part of the oxyanion hole that activates the reaction and also to activate water that comes in further along the reaction cycle. (FIG. 14).

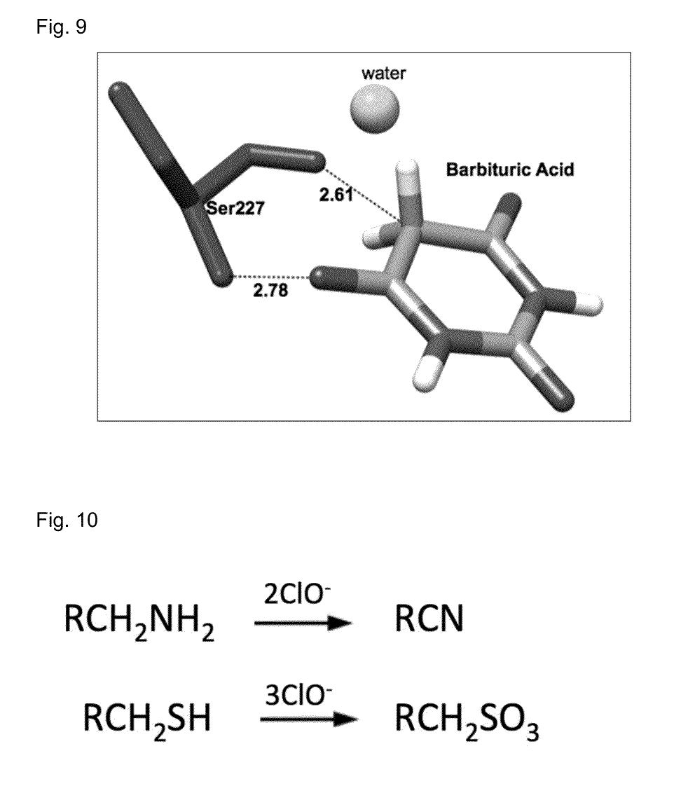

Overall, the data indicate that CAH carries out catalysis by Lys156 activating Ser226 that serves as a nucleophile to attack a carbonyl carbon of cyanuric acid. This forms a serine ester intermediate that can be readily hydrolyzed by an activated water to yield the products of the cyanuric acid hydrolase catalyzed reaction. FIG. 9 shows the active site configuration of the serine poised for attack on substrate but in this case not reacting with an unreactive methylene carbon of barbituric acid, that is bound as a stable surrogate for cyanuric acid.

Knowledge of the structure and the active site allows one to probe sites of reaction of chlorine (e.g., hypochlorite and TCYA) that could destroy cyanuric acid hydrolase activity. In some cases, amino acids on the surface or in other places outside the active site might react and perturb the enzyme structure, thus leading to loss of activity. Those non-essential amino acids can be changed to amino acids that would not react with chlorine, thus diminishing the susceptibility of the enzyme to chlorine inactivation.

In one aspect, therefore, this disclosure describes a composition that includes a cell that is at least partially covered with a matrix material. The matrix material can be a silica gel or silica sol that possesses at least one chemical group that can react with bleach (e.g., hypochlorite, chlorine, trichloroisocyanuric acid, etc.).

The cell can be any cell that naturally expresses an enzyme that degrades cyanuric acid. Alternatively, the cell can be a recombinant cell that has been genetically modified to exhibit increased degradation of cyanuric acid compared to a wild-type control. In some embodiments a genetically modified host cell can be an E. coli cell. In other embodiments, however, a recombinant cell can be constructed, and the methods of making and using the recombinant cells can be performed, using any suitable host cell. Thus, a recombinant cell can be, or be derived from, any suitable microbe including, for example, a prokaryotic microbe or a eukaryotic microbe. As used herein, the term “or derived from” in connection with a microbe simply allows for the “host cell” to possess one or more genetic modifications before being modified to exhibit increased cyanuric acid degradation enzymatic activity. Thus, the term “recombinant cell” encompasses a “host cell” that may contain nucleic acid material from more than one species before being modified to exhibit cyanuric acid degradation activity.

In some embodiments, the host cell may be selected to possess one or more natural physiological activities. For example, the host cell may be photosynthetic (e.g., cyanobacteria) or thermophilic (e.g., Moorella thermoacetica).

In some embodiments, the host cell may be, or be derived from, a eukaryotic microbe such as, for example, a fungal cell. In some of these embodiments, the fungal cell may be, or be derived from, a member of the Saccharomycetaceae family such as, for example, Saccharomyces cerevisiae.

In other embodiments, the host cell may be, or be derived from, a prokaryotic microbe such as, for example, a bacterium. In some of these embodiments, the bacterium may be a member of the phylum Protobacteria. Exemplary members of the phylum Protobacteria include, for example, members of the Enterobacteriaceae family (e.g., Escherichia coli) and, for example, members of the Pseudomonaceae family (e.g., Pseudomonas putida). In other cases, the bacterium may be a member of the phylum Firmicutes. Exemplary members of the phylum Firmicutes include, for example, members of the Bacillaceae family (e.g., Bacillus subtilis), members of the Clostridiaceae family (e.g., Clostridium cellulolyticum) and, for example, members of the Streptococcaceae family (e.g., Lactococcus lactis). In other cases, the bacterium may be a member of the phylum Cyanobacteria.

In some embodiments, the host cell may be a cell selected because it naturally exhibits some resistance to chlorine. In other embodiments, the host cell may be genetically modified to produce one or more heterologous metabolites—e.g., glutathione—that can protect the cell against chlorine.

In some embodiments, the host cell can exhibit increased cyanuric acid degradation activity compared to a wild-type control. As used herein, the terms “activity” with regard to particular cyanuric acid degrading enzyme refers to the ability of a polypeptide, regardless of its common name or native function, to degrade cyanuric acid, regardless of whether the “activity” is less than, equal to, or greater than the native activity of the identified enzyme. Exemplary methods for measuring the cyanuric degradation activity of an enzyme are described in the EXAMPLES section, below.

As used herein, an increase in enzymatic activity can be quantitatively measured and described as a percentage of the enzymatic activity of an appropriate wild-type control. The enzymatic activity exhibited by a genetically-modified cell or polypeptide can be, for example, at least 110%, at least 125%, at least 150%, at least 175%, at least 200% (two-fold), at least 250%, at least 300% (three-fold), at least 400% (four-fold), at least 500% (five-fold), at least 600% (six-fold), at least 700% (seven-fold), at least 800% (eight-fold), at least 900% (nine-fold), at least 1000% (10-fold), at least 2000% (20-fold), at least 3000% (30-fold), at least 4000% (40-fold), at least 5000% (50-fold), at least 6000% (60-fold), at least 7000% (70-fold), at least 8000% (80-fold), at least 9000% (90-fold), at least 10,000% (100-fold), or at least 100,000% (1000-fold) of the activity of an appropriate wild-type control.

Alternatively, an increase in enzymatic activity may be expressed as at an increase in k_{cat} such as, for example, at least a two-fold increase, at least a three-fold increase, at least a four-fold increase, at least a five-fold increase, at least a six-fold increase, at least a seven-fold increase, at least an eight-fold increase, at least a nine-fold increase, at least a 10-fold increase, at least a 15-fold increase, or at least a 20-fold increase in the k_{cat} value of the enzymatic conversion.

An increase in enzymatic activity also may be expressed in terms of a decrease in Kₘ such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the Kₘ value of the enzymatic conversion.

A decrease in enzymatic activity can be quantitatively measured and described as a percentage of the enzymatic activity of an appropriate wild-type control. The enzymatic activity exhibited by a genetically-modified cell or polypeptide can be, for example, no more than 95%, no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 3%, no more than 2%, no more than 1% of the activity, or 0% of the activity of a suitable wild-type control.

Alternatively, a decrease in enzymatic activity can be expressed as an appropriate change in a enzymatic constant. For example, a decrease in enzymatic activity may be expressed as at a decrease in k_{cat} such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the k_{cat} value of the enzymatic conversion.

A decrease in enzymatic activity also may be expressed in terms of an increase in Kₘ such as, for example, an increase in Kₘ of at least two-fold, at least three-fold, at least four-fold, at least five-fold, at least six-fold, at least seven-fold, at least an eight-fold, at least nine-fold, at least 10-fold, at least 15-fold, at least 20-fold, at least 25-fold, at least 30-fold, at least 35-fold, at least 40-fold, at least 45-fold, at least 50-fold, at least 75-fold, at least 100-fold, at least 150-fold, at least 200-fold, at least 230-fold, at least 250-fold, at least 300-fold, at least 350-fold, or at least 400-fold.

In some case, the cell can be a cell that has been evolved in the laboratory to become more resistant to chlorine damage. The cells may be evolved under selective pressure of growth on cyanuric acid or other chlorine source.

Without wishing to be bound by any particular theory of operation, the cell membrane can be permeable to cyanuric acid, thereby allowing cyanuric acid to contact the cyanuric acid-degrading enzyme inside the cell. The cell membrane can also protect the cyanuric acid-degrading enzyme from bleach that would otherwise inactivate the enzyme.

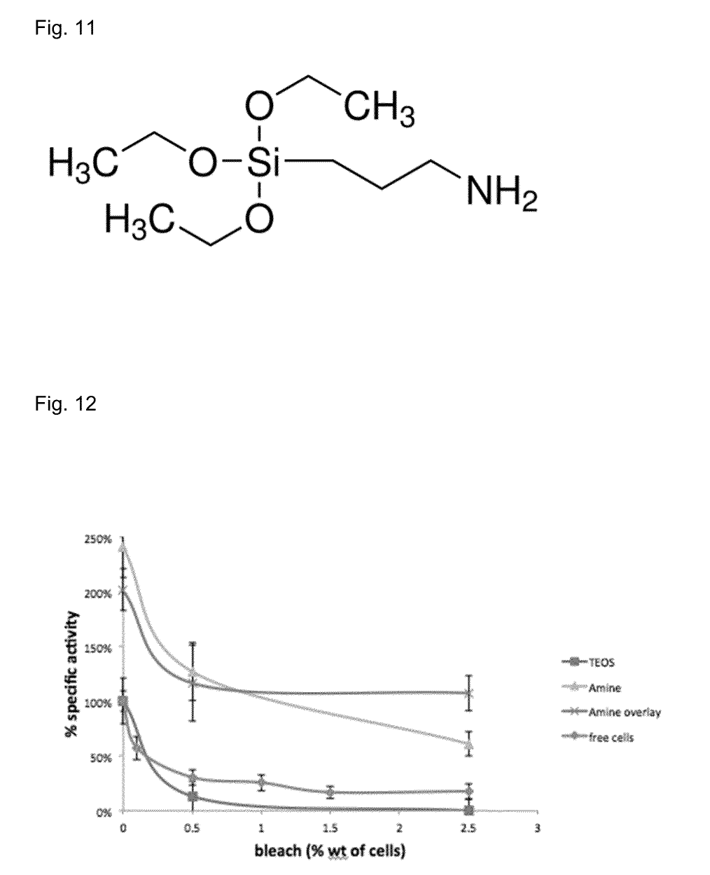

The cell is at least partially covered with a silica matrix material. In some exemplary embodiments, the cell may be encapsulated by the silica matrix material. While occasionally discussed above in the context of exemplary embodiments in which the cell is encapsulated by the silica matric material, certain properties of those exemplary compositions are not dependent upon the cells being encapsulated. FIG. 12 shows that cells that are partially covered (Amine overlay) with an amine-containing silica matrix material is at least as effective as at maintaining cyanuric acid-degrading enzyme activity as cells encapsulated in the same material (Amine).

In some embodiments, therefore, cyanuric acid degradation activity can be maintained by modifying the silica matrix material. FIG. 12 shows that one can use modified gels to further protect cyanuric acid-degrading enzymatic activity beyond the protection afforded by the cell environment. While described herein in the context of exemplary embodiments in which the matrix material includes a silica material (e.g., APTES), the compositions, devices, and methods described herein can be prepared or practiced, as the case may be, using alternative matrix materials. Exemplary alternative matrix materials include, for example, any material that provides an organic functional group that is reactive with bleach, hypochlorite, or other chlorinating agent so that the chlorine reacts preferentially with the organic group rather than with the enzyme. Organic functional groups that protect in this manner can include, for example, an amine, a thiol, a thioether, a phenolic group, an alkene, a ketone, an alkoxyphenol ether, or another organic functional group that reacts with hypochlorite, or chlorine. Exemplary materials include wood sawdust (which contains alkoxy phenolic ethers), rubber tire material (which contains sulfur groups as part of the rubber vulcanization process), and activated carbon.

Silica matrix materials prepared from 3-aminopropyltriethoxysilane (APTES), a gel material with an amine functionality, were evaluated for their ability to protect cyanuric acid hydrolase activity in the presence of bleach. The structure of APTES is shown in FIG. 11. Gels were prepared using APTES or TEOS and then used to encapsulate E. coli that expressed Moorella CAH enzyme (Example 3). The gels were tested with a challenge of hypochlorite known to damage CAH activity acutely. A range of hypochlorite concentrations were tested. The results of these experiments are shown in FIG. 12.

The amine-containing matrix materials preserved a greater portion of cyanuric acid hydrolase activity than did the TEOS matric materials. APTES matrix materials can react with hypochlorite ion based on the chemistry of bleach illustrated in FIG. 10. Thus, as mentioned above, the APTES matrix material was effective at preserving CAH activity against chlorine inactivation whether the APTES matrix material fully encapsulated cells (FIG. 12, Amine) or partially covered cells (FIG. 12, Amine overlay).

This matrix-chemistry-based protection can enhance the cell-based protection methods described above. Thus, a device that includes a chlorine reactive group can provide two separate strategies for preserving the enzymatic activity that breaks down cyanuric acid even in an application or environment with high chlorine content. This allows continuous cyanuric acid removal in the presence of hypochlorite, allowing optimum disinfection for protecting pools, spas, fountains and other waters from viral, bacterial, and/or parasitic infectious agents.

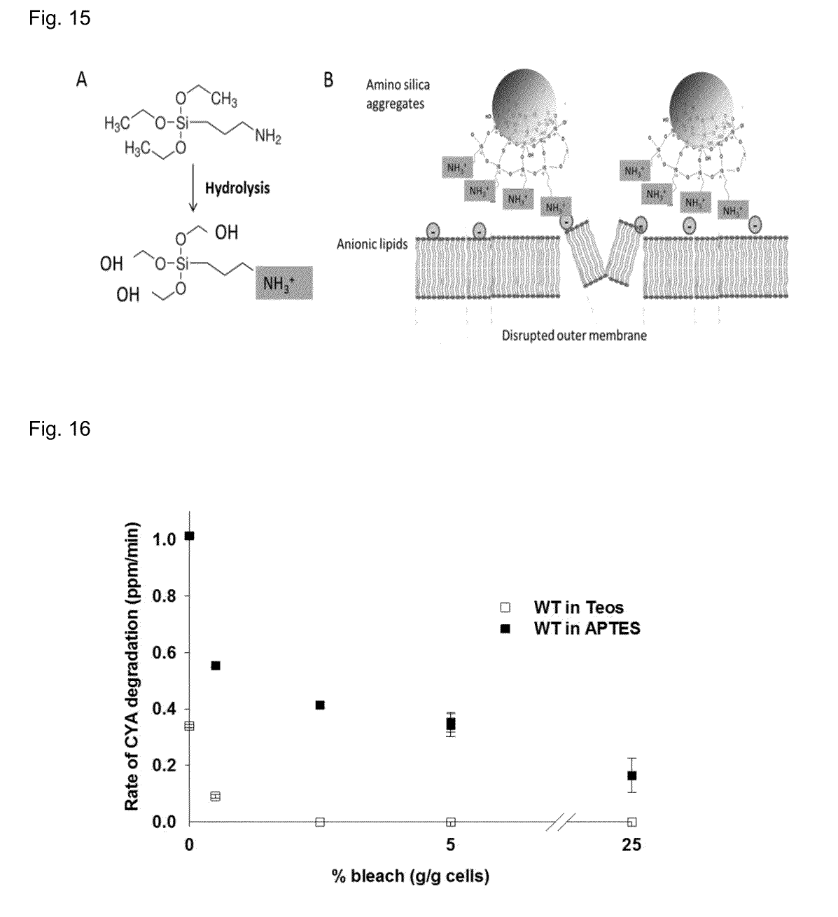

In some cases, the matrix material can interact with the cell membrane to selectively increase permeability of the cell, as illustrated in FIG. 15. The result can be a change in the structure and/or integrity of the cell membrane that can lead to the cell membrane being more permeable to small molecules but remain impermeable to larger molecules such as, for example, the cyanuric acid-degrading enzyme.

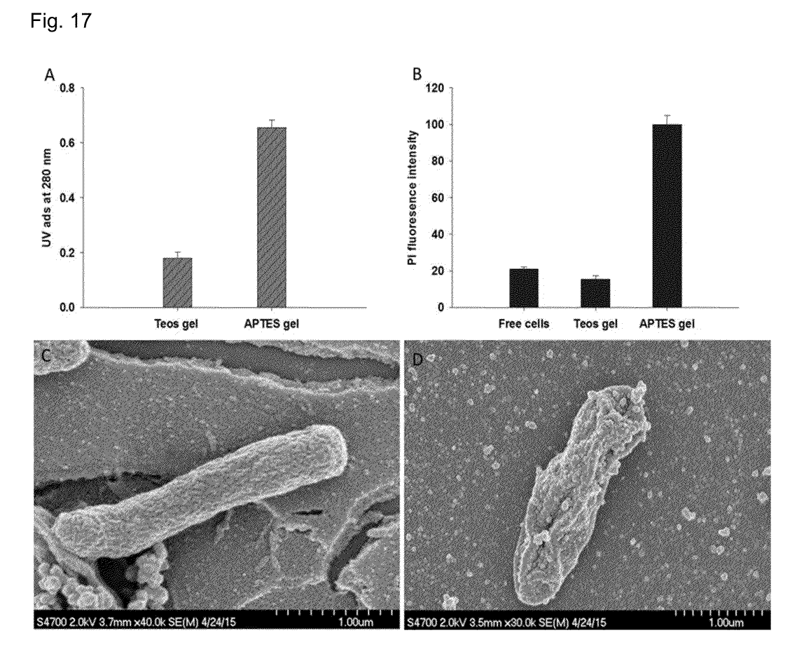

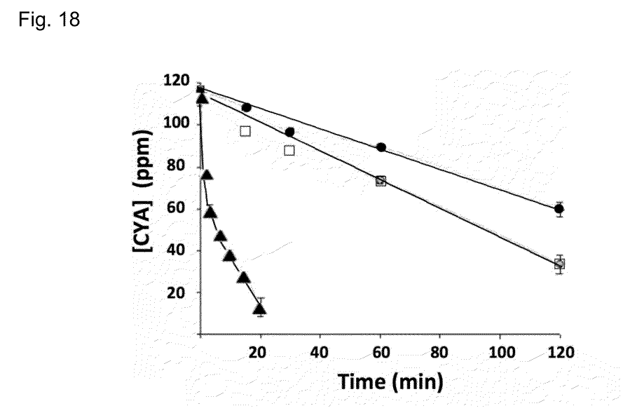

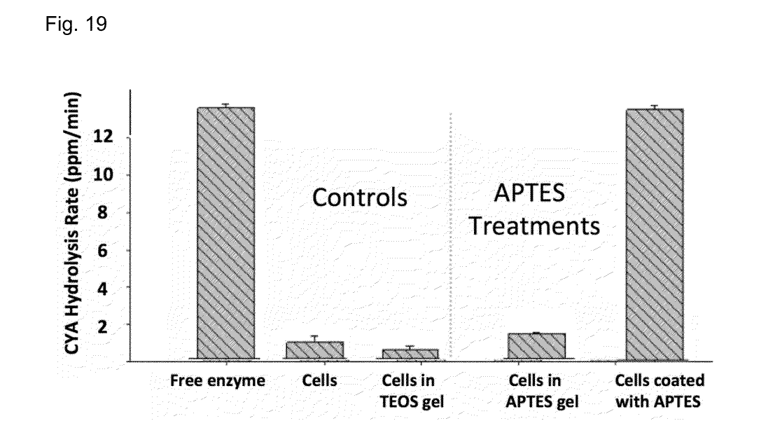

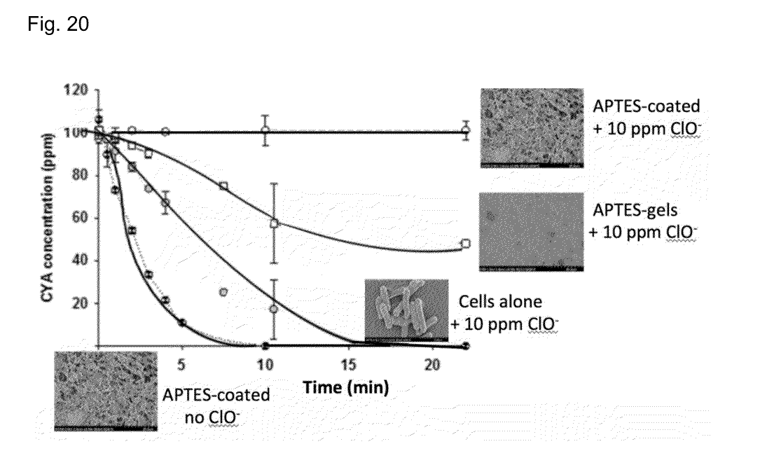

FIG. 17A and FIG. 17B illustrate that a cell at least partially covered with an exemplary amine-containing matric material (APTES) exhibits increased permeability to small molecules. To assess permeability, the TEOS silica gel and APTES silica gel were left to shake after preparation with 3 mL of PBS. The solutions were then monitored for absorbance by UV-Vis spectrometer at 280 nm. The absorbance of the solution washed off the amino-silica gel had significantly higher readings, suggesting that more organic molecules were leaking out (FIG. 17A). Whole E. coli cells were mixed with the two precursors (APTES and TEOS) for 10 minutes, then the solutions were then centrifuged and the pellet was re-dispersed in PBS and the permeability probe propidium iodide was added. Propidium iodide fluoresces intensely when it is in contact with the cellular DNA and can only enter the cell if the membrane is compromised. Thus, propidium iodide fluorescence is commonly used to distinguish live intact cells from permeabilized cells. The fluorescence intensity of propidium iodide was measured at 535 nm excitation and 617 nm emission (DNA-bound fluorescence). FIG. 17B shows that the APTES-encapsulated cells were more permeable than TEOS-encapsulated cells or untreated cells.

Thus, certain matric materials can modify the permeability of the cell, allowing cyanuric acid to more readily enter the cell, where it can be degraded by the enzyme that is retained within the cell. FIG. 17D shows the morphology of a cell after having been removed from an APTES gel matrix. The morphology is distinctly different than the morphology of a similar cell that has been removed from a TEOS gel matrix (FIG. 17C).

FIG. 16 provides data demonstrating that a result of increasing the permeability of the cell and/or altering the morphology of the cell can result in an increase in enzyme activity in the presence of bleach. Cells encapsulated in an exemplary APTES silica gel exhibited an increased rate of cyanuric acid degradation compared to a silica-based matrix material that does not include a chemical group—e.g., an amine or thiol group—that can interact with the cell membrane and/or react with bleach.

While described above in the context of an exemplary embodiment in which the matrix material is an amine-containing matrix material, 3-aminopropyltriethoxysilane (APTES), the compositions, devices, and methods described can involve any suitable chlorine-reactive gel material. Exemplary alternative chlorine-reactive gel materials include, for example vinyl siloxanes, phenyl siloxanes, cyclic aza siloxanes, cyclic thia siloxanes. Protection also can be accomplished by connecting auxiliary polymers to the siloxane groups where the polymers reaction with chlorine and thus reduce the extent to which chlorine can diminish CAH activity inside cells. A non-exhaustive list of exemplary silica polymer precursors that possess amino or thio organic functional groups includes, for example, (3-aminopropyl)triethoxysilane (APTES), (3-aminopropyl)trimethoxysilane, (4-aminobutyl)triethoxysilane, aminophenyltrimethoxysilane, p-aminophenyltrimethoxysilane, m-aminophenyltrimethoxysilane, 3-aminopropyltris(methoxyethoxy)silane, 11-aminoundecyltriethoxysilane, 2-(4-pyridylethyl)triethoxysilane, 2-(trimethoxysilylethyl)pyridine, N-(3-trimethoxysilylpropyl)pyrrole, 3-(m-aminophenoxy)propyl trimethoxysilane, 3-aminopropyldiisopropylethoxysilane, 3-aminopropyldimethylethoxysilane, N-(2-aminoethyl)-3-aminopropyltrimethoxysilane, (3-trimethoxysilylpropyl)diethylenetriamine, N-butylaminopropyltrimethoxysilane, N-ethylaminoisobutyltrimethoxysilane, N-methylaminopropyltrimethoxysilane, N-phenylaminopropyltrimethoxysilane, 3-(N-allylamino)propyltrimethoxysilane, (cyclohexylaminomethyl)triethoxysilane, N-cyclohexylaminopropyltrimethoxysilane, N-ethylaminoisobutylmethyldiethoxysilane, and (phenylaminomethyl)methyldimethoxysilane.

In another aspect, this disclosure describes a device that includes a cell at least partially covered with a matrix material as described herein. In some embodiments, the device can include a surface configured to contact at least a portion of a water system and at least partially coated with the any embodiment of the silica matrix composition described herein. The device can include, for example, a filtration material having a surface at least partially coated with the silica matrix composition. In such embodiments, the device can simultaneously provide filtration and removal of cyanuric acid and, thus, provide a combination of filtration and increased disinfectant activity.

Alternative exemplary devices include devices that include a water permeable chamber such as, for example, canister, a column, a permeable bag, or any device having a surface configured to have at least a portion of a water system run over, past, and/or through the chamber.

In another aspect, therefore, the compositions and devices described above may be used in a method that involves removing cyanuric acid from a sample. Generally, the method includes contacting the sample with any embodiment of the compositions and devices described herein, then allowing the enzyme to degrade cyanuric acid in the sample. The design of the compositions and devices allows the removal of cyanuric acid to be performed on a continuous basis rather than on a batch basis.

In embodiments in which the silica matrix composition is a component of a device, the method can include simply placing the device in contact with the water system in a manner effective to allow at least a portion of the water system to contact the silica matrix composition. In many embodiments, this can include conventional installation of an otherwise conventional device (e.g., a filter) that has been modified to include at least one surface at least partially coated with the silica matrix composition. In some embodiments, the composition may be in the form of a dry formulation or a suspension that includes the silica matrix composition. In such embodiments, the method can simply include introducing the dry formulation or the suspension into the water system.

In some embodiments, the sample can include chlorinated water. Water in the sample may be chlorinated by the addition of one or more of hypochlorite, hypochlorous acid, and/or N-chlorinated compounds of any type (e.g., trichloroisocyanuric acid and/or dichloroisocyanuric acid).

While described above in the context of an exemplary embodiment in which the compositions, devices, and methods involve removing cyanuric acid from a water system, the compositions, devices, and methods described herein can involve removing cyanuric acid from any fluid sample. Thus, description of the compositions, devices, and methods in the context of removing cyanuric acid from a water system such as, for example, swimming pools, spas, fountains, wastewater treatment, or fisheries is purely exemplary and nonlimiting.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Silica gel having cells encapsulated in TEOS (TEOS gel) was prepared generally as described in International Publication No. WO 2012/116031 A2.

Silica gel having cells encapsulated in APTES (APTES gel) was prepared generally as described in International Publication No. WO 2012/116031 A2, except that 3-aminopropyltriethoxysilane (APTES) was used as the silica gel precursor rather than TEOS. Generally, the APTES gel was prepared by adding 1.5 mL of APTES to 8.5 mL of water plus HCl (40 μL, pH=2-2.5) and stirred on ice for two hours. 1.75 mL of SiO₂ particles (NP-125-40; 80 nm, 400 g/L) were mixed with 250 μL CAH-containing cells 0.1 g/mL and placed on ice. 250 μL of the APTES (completely hydrolyzed) solution was added to the particles and cells and the gel was allowed to cure for one hour.

A TEOS gel surface covered with APTES was prepared by first preparing a TEOS gel. The TEOS gel was prepared by adding 1.75 mL SiO₂, 250 μL cells, and 250 μL TEOS, then allowing the gel to cure for one hour. 300 μL APTES, enough to cover the entire surface of the TEOS gel, was added and left for 30 minutes at room temperature. The APTES-coated TEOS gels were rinsed with phosphate buffered saline.

### Assays Used to Measure Cyanuric Acid Hydrolase Activity

### Assay 1:

A coupled-protein assay was developed as a highly sensitive method to measure CAH activity. Biocatalysts were incubated with 0.5 ml of 10 mM cyanuric acid in 0.1 M potassium phosphate buffer (pH 7) for time periods ranging from 0.5-65.0 h. The CAH reactions were stopped at four discrete time points by boiling the reaction tubes for ten minutes. Next, the reaction tubes were cooled to room temperature, a 5 μg aliquot of purified biuret hydrolase was added to each tube, and then the tubes were incubated at room temperature for one hour. After incubation, ammonia was quantitated colorimetrically via the Berthelot reaction (Weatherburn, M. W., 1967, Anal. Chem. 39:971-974). Specific activity of the mutant CAHs was calculated at each time point based on 1 mole ammonia/1 mole biuret, and 1 mole biuret/1 mole cyanuric acid cleaved. Control samples without enzyme(s) were incubated in parallel to determine background levels of cyanuric acid hydrolysis or ammonia release. All samples were analyzed in triplicate.

### Assay 2:

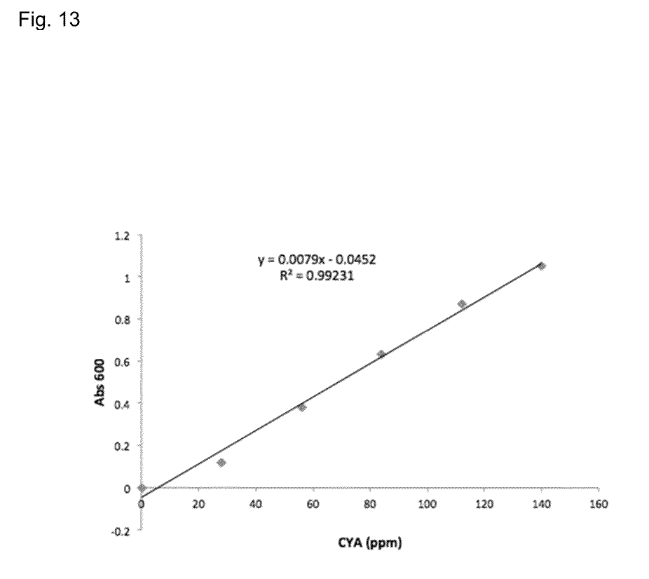

20 mM dissolved melamine (Sigma-Aldrich, St. Louis, Mo.) was combined with cyanuric acid in a 1:1 molar ratio. Melamine complexation with cyanuric acid was measured as turbidity (light scattering) at a wavelength of 600 nm in a spectrophotometer. This provides a standard curve (FIG. 13) superior to a conventional commercial assay kit (ColorQ Pro 7, LaMotte Co., Chestertown, Md.) and more accurately quantifies cyanuric acid and was used to monitor disappearance of cyanuric acid.

### Example 2

### Mutational Study

Site directed mutagenesis was conducted with a QuikChange kit (Agilent Technologies, Santa Clara, Calif.), using the primers as indicated in Table 1.

TABLE 1
Primers Used

| Site- | | |
|---|---|---|
| directed | | |
| mutation | Primers | |
| S79A | 5′-gcctcgtcatggccggcggcacc-3′ | 5′-ggtgccgccggccatgacgaggc-3′ |
| | (SEQ ID NO: 1) | (SEQ ID NO: 2) |
| S226A | 5′-gcgcgcgcgagctgtgccagcggt-3′ | 5′-accgctggcacagctcgcgcgcgc-3′ |
| | (SEQ ID NO: 3) | (SEQ ID NO: 4) |
| S333A | 5′-acggagatctatgtcgccggcggcggc-3′ | 5′-gccgccgccggcgacatagatctccgt-3′ |
| | (SEQ ID NO: 5) | (SEQ ID NO: 6) |
| K40A | 5′-cctcgccatctaggagcgaccgagggcaatggc-3′ | 5′-gccattgcccteggtcgctccaaagatggcgagg-3′ |
| | (SEQ ID NO: 7) | (SEQ ID NO: 8) |
| K156A | 5′-gcatttcgtgcaggtggcatgcccgcttctcacc-3′ | 5′-ggtgagaagcgggcatgccacctgcacgaaatgc-3′ |
| | (SEQ ID NO: 9) | (SEQ ID NO: 10) |
| K285A | 5′-catcgtgctcgccgcggeggagcccagc-3′ | 5′-gctgggctccgccgcggcgagcacgatg-3′ |
| | (SEQ ID NO: 11) | (SEQ ID NO: 12) |
| R188K | 5′-ctcaaatccatgggcctctcaaagggggcgagcgc-3′ | 5′-gcgctcgccccattgagaggcccatggatttgag-3′ |
| | (SEQ ID NO: 13) | (SEQ ID NO: 14) |
| R188Q | 5′-tgggcctctcacagggggcgagcgcg-3′ | 5′-cgcgctcgccccctgtgagaggccca-3′ |
| | (SEQ ID NO: 15) | (SEQ ID NO: 16) |

The protein yield of both wild type and mutant enzymes was 7-10 mg/L. CD spectroscopy experiments were conducted over the range of 200-250 nm on a J-815 CD spectrophotometer equipped with a Peltier temperature control (Jasco Products Co., Oklahoma City, Okla.). The wild-type and mutant enzymes were analyzed by circular dichroism (CD) and indicated that the proteins had the correct secondary structure and were stable. The wild type and mutant enzymes were assayed.

### Example 3

To construct E. coli strains containing cyanuric acid hydrolase, pET28b+::Moth 2120, pET28b+::atzD and pET2828b+;;trzD (each of which is described in U.S. Pat. No. 8,367,389) were used as the PCR template. The full length of each gene was amplified via PCR using the primers AtzD-F, AtzD-R, TrzD-F and TrzD-R. The fragments were then cloned into the EcoRI and NotI cloning sites of the pUCMod vector, yielding pUCMod atzD and pUCMod trzD (Table 2). The plasmids were introduced into E. coli DH5α by electroporation. E. coli DH5α competent cells were prepared by washing cells harvested at the exponential phase (OD₆₀₀˜0.5) with distilled water and a 10% (v/v) glycerol solution.

The coding region from Moorella thermoacetica ATCC 39073 was amplified from pET28b+::Moth_{—}2120 with the primers CAH-F and CAH-R. The fragment was cloned into the EcoRI and NcoI cloning sites of the STRATACLONE PCR cloning vector (Agilent Technologies, Inc., Santa Clara, Calif.). The resulting plasmid was digested with the same restriction enzymes and the fragment released from the STRATACLONE plasmid was ligated into pUCMod, yielding pUCMod CAH (Table 2). The plasmid was introduced into MAX Efficiency E. coli DH5α Competent Cells (Life Technologies, Carlsbad, Calif.).

TABLE 2
Strains, plasmids, and primers

| | Relevant markers and characteristic^{a} | |
|---|---|---|
| Strain | | |
| DH5α | Δ(lacZYA-argF)U169 (Φ80lacZ ΔM15) | |
| CAH | Strain DH5α harboring pUCMod CAH; Amp^{R} | |
| AtzD | Strain DH5α harboring pUCMod atzD; Amp^{R} | |
| TrzD | Strain DH5α harboring pUCMod trzD; Amp^{R} | |
| Plasmid | | |
| pUCMod | rep (pMB1), bla (Amp^{R}), constitutive lac promoter | |
| pUCMod | pUCMod carrying the Moorella thermoacetica ATCC 39073 | |
| CAH | Cyanuric Acid Hydrolase coding region | |
| pUCMod | pUCMod carrying the Pseudomonas sp. strain ADP atzD | |
| AtzD | coding region | |
| pUCMod | pUCMod carrying the Acidovorax avenae subsp. citrulli | |
| TrzD | trzD coding region | |
| Primers^{b} | Sequence 5′→3′ | SEQ ID NO: |
| CAH-F | GAATTCAGGAGGATTACAAAATGCAAAAAGTCTTTCGTATCCCA | 17 |
| | ACAG | |
| CAH-R | ATTACCATGGCTACACCCTGGCAATAACAGCAATTGGG | 18 |
| AtzD-F | ATTGAATTCAGGAGGATTACAAAATGTATCACATCGACGTTTTC | 19 |
| | CGAATCCCTTGCCAC | |
| AtzD-R | ATTTAATGCGGCCGCTTAAGCGCGGGCAATGAC | 20 |
| TrzD-F | ATTGAATTCAGGAGGATTACAAAATGCAAGCGCAAGTTTTTCGA | 21 |
| | GTTCC | |
| TrzD-R | ATTTAATGCGGCCGCTTAAGCTGTGCGCGCGATAAC | 22 |

^{a}Amp^{R}; resistance to ampicillin.
^{b}Underlined letters indicate restriction enzyme recognition sites. Bold letters indicate a Shine-Dalgarno sequence.

### Example 4

To assess permeability, the TEOS silica gel and APTES silica gel were prepared as described in Example 1. The gels were left to shake after preparation with 3 mL of PBS. The solutions were then monitored for absorbance by UV-Vis spectrometer at 280 nm. The absorbance of the solution washed off the amino-silica gel had significantly higher readings, suggesting that more organic molecules were leaking out. Results are shown in FIG. 17A.

Whole E. coli cells were mixed with the two precursors (APTES and TEOS) for 10 minutes. The solutions were then centrifuged and the pellet was re-dispersed in PBS and the permeability probe propidium iodide (PI) was added. PI fluoresces intensely when in contact with the cellular DNA and can only enter the cell if the membrane is compromised. The fluorescence intensity of PI was measured at 535 nm excitation and 617 nm emission (DNA-bound fluorescence). Results are shown in FIG. 17B.

Scanning electron microscopy (SEM) measurements: E. coli cells mixed with TEOS and APTES precursors were pipetted on to a small aluminum slide. The slides were dipped in 2.5% gluteraldehyde for three hours and then gradually dehydrated in a series of ethanol washes (50%, 70%, 80%, 95% and 100% EtOH, five minutes each wash). The ethanol was then evaporated overnight. Finally, the dried gels mounted on the slides were placed on a SEM carrier and sputter-coated with a thin layer of gold-palladium. SEM images were taken by a Hitachi 54700 machine (Hitachi High Technologies America, Inc., Hillsboro. Oreg.). Results are shown in FIG. 17C and FIG. 17D.

### Example 5

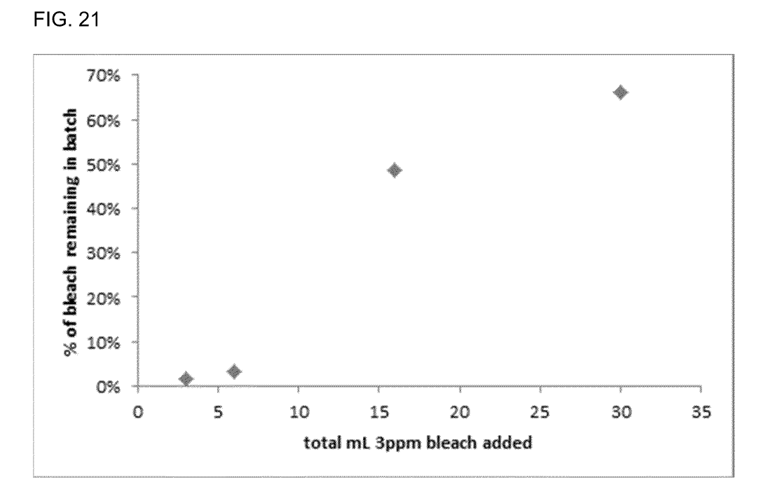

A 0.1 g plug of crumb rubber was placed into a narrow column. The plug was approximately 0.5 inches tall. 3 mL of a bleach solution containing 3 ppm hypochlorite was passed through the column. The flow through was collected and the bleach concentration was measured to determine the fraction of bleach remaining in the flow through. The process was repeated with a second 3 mL of bleach solution, then again with 10 mL of bleach solution, and finally with 14 mL of bleach solution. Results are shown in FIG. 21.

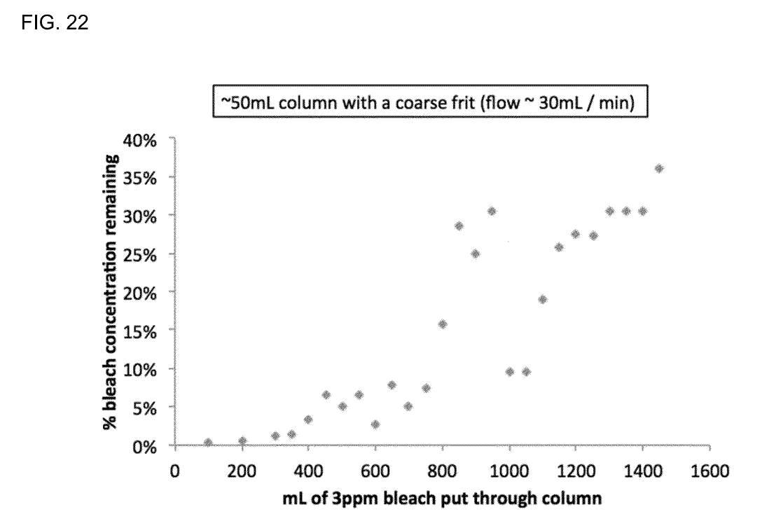

The experiment was repeated using cherry wood sawdust. Results are shown in FIG. 22.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A composition comprising:
a cell that expresses at least one enzyme that degrades cyanuric acid; and
a silica matrix material covering at least a portion of the cell, wherein the silica matrix material comprises at least one chlorine-reactive chemical group.

2. The composition of claim 1 wherein the cell is genetically-modified to exhibit an increase in cyanuric acid degradation activity compared to a wild-type control.

3. The composition of claim 1 wherein the cell is an E. coli cell.

4. The composition of claim 1 wherein the enzyme comprises a cyanuric amidohydrolase.

5. The composition of claim 1 wherein the enzyme comprises a cyanuric acid hydrolase.

6. The composition of claim 1 wherein the chlorine-reactive chemical group comprises and amine group or a thiol group.

7. The composition of claim 6 wherein the chlorine-reactive chemical group is a constituent of a silica precursor from which the matrix material is prepared.

8. The composition of claim 1 wherein the chlorine-reactive chemical group is a constituent of an additive.

9. The composition of claim 8 wherein the additive comprises sawdust or activated charcoal.

10. A device comprising the composition of claim 1.

11. The device of claim 10 comprising a surface at least partially coated with the composition of claim 1 and configured to contact at least a portion of a water system.

12. The device of claim 11 wherein the device comprises a filter.

13. The device of claim 10 comprising a water-permeable chamber that contains the composition of claim 1.

14. The device of claim 13 wherein the chamber comprises a canister, a column, or a bag.

15. A method for removing cyanuric acid from a sample, the method comprising:
contacting the sample with a composition comprising:
a cell that expresses at least one enzyme that degrades cyanuric acid; and
a silica matrix material covering at least a portion of the cell, wherein the silica matrix material comprises at least one chlorine-reactive chemical group; and
allowing the enzyme to degrade cyanuric acid in the sample.

16. The method of claim 15 wherein the sample comprises chlorinated water.

17. The method of claim 16 wherein the sample comprises hypochlorite ion.

18. The method of claim 15 wherein contacting the sample with the composition comprises placing a device comprising the composition in contact with the sample.

19. The method of claim 15 wherein:
the composition comprises a dry formulation; and
contacting the composition with the sample comprises introducing the dry formulation directly to the sample.

20. A device for removing cyanuric acid from a fluid sample, the device comprising:
a surface; and
a composition coating at least a portion of the surface, the composition comprising:
a cell that expresses at least one enzyme that degrades cyanuric acid; and
a silica matrix material covering at least a portion of the cell.

21. The device of claim 20 wherein the silica matrix material comprises at least one chlorine-reactive chemical group.

22. The device of claim 20 wherein the surface is configured to contact the fluid sample.


SEQUENCE LISTING (22 sequences)

SEQ ID NO: 1 - DNA, 23 bp, Artificial
  Primer
gcctcgtcatggccggcggcacc

SEQ ID NO: 2 - DNA, 23 bp, Artificial
  Primer
ggtgccgccggccatgacgaggc

SEQ ID NO: 3 - DNA, 24 bp, Artificial
  Primer
gcgcgcgcgagctgtgccagcggt

SEQ ID NO: 4 - DNA, 24 bp, Artificial
  Primer
accgctggcacagctcgcgcgcgc

SEQ ID NO: 5 - DNA, 27 bp, Artificial
  Primer
acggagatctatgtcgccggcggcggc

SEQ ID NO: 6 - DNA, 27 bp, Artificial
  Primer
gccgccgccggcgacatagatctccgt

SEQ ID NO: 7 - DNA, 34 bp, Artificial
  Primer
cctcgccatctttggagcgaccgagggcaatggc

SEQ ID NO: 8 - DNA, 34 bp, Artificial
  Primer
gccattgccctcggtcgctccaaagatggcgagg

SEQ ID NO: 9 - DNA, 34 bp, Artificial
  Primer
gcatttcgtgcaggtggcatgcccgcttctcacc

SEQ ID NO: 10 - DNA, 34 bp, Artificial
  Primer
ggtgagaagcgggcatgccacctgcacgaaatgc

SEQ ID NO: 11 - DNA, 28 bp, Artificial
  Primer
catcgtgctcgccgcggcggagcccagc

SEQ ID NO: 12 - DNA, 28 bp, Artificial
  Primer
gctgggctccgccgcggcgagcacgatg

SEQ ID NO: 13 - DNA, 35 bp, Artificial
  Primer
ctcaaatccatgggcctctcaaagggggcgagcgc

SEQ ID NO: 14 - DNA, 35 bp, Artificial
  Primer
gcgctcgccccctttgagaggcccatggatttgag

SEQ ID NO: 15 - DNA, 26 bp, Artificial
  Primer
tgggcctctcacagggggcgagcgcg

SEQ ID NO: 16 - DNA, 26 bp, Artificial
  Primer
cgcgctcgccccctgtgagaggccca

SEQ ID NO: 17 - DNA, 48 bp, Artificial
  Primer
gaattcaggaggattacaaaatgcaaaaagtctttcgtatcccaacag

SEQ ID NO: 18 - DNA, 38 bp, Artificial
  Primer
attaccatggctacaccctggcaataacagcaattggg

SEQ ID NO: 19 - DNA, 59 bp, Artificial
  Primer
attgaattcaggaggattacaaaatgtatcacatcgacgttttccgaatcccttgccac

SEQ ID NO: 20 - DNA, 33 bp, Artificial
  Primer
atttaatgcggccgcttaagcgcgggcaatgac

SEQ ID NO: 21 - DNA, 49 bp, Artificial
  Primer
attgaattcaggaggattacaaaatgcaagcgcaagtttttcgagttcc

SEQ ID NO: 22 - DNA, 36 bp, Artificial
  Primer
atttaatgcggccgcttaagctgtgcgcgcgataac